(19) United States
(12) Patent Application Publication
(10) Pub. No.: US 2026/0354882 A1
(43) Pub. Date: Nov. 12, 2026

(54) SYSTEMS AND/OR METHODS FOR HEATING AND/OR COOLING A CRYOGENIC NEEDLE

(71) Applicant: UC CARE LTD, Yokneam (IL)

(72) Inventors: Yeshayahu SCHATZBERGER, Haifa (IL); Yoram KEDEM, Haifa (IL); Ichai PEREZ, Haifa (IL); Keren SCHWEITZER, Tal-El (IL); Benjamin LUIKERT THOMAS, Netanya (IL)

(21) Appl. No.: 19/486,951

(22) PCT Filed: May 23, 2024
(86) PCT No.: PCT/IB2024/055012

§ 371 Date: Nov. 23, 2025

Related U.S. Application Data
(60) Provisional application No. 63/469,466, filed on May 29, 2023.

(51) Int. Cl. A61B 18/02; A61B 17/00; A61B 18/00

(52) U.S. Cl. CPC A61B 18/02; A61B 2017/00199; A61B 2018/00077; A61B 2018/00101; A61B 2018/00172; A61B 2018/00702; A61B 2018/00714; A61B 2018/0262; A61B 2018/0293

(57) ABSTRACT

A system for affecting a temperature of a cryogenic needle includes an optical fiber and a radiation system. The radiation system is used for emitting radiation through the optical fiber towards the needle's distal tip.

### TECHNICAL FIELD

Embodiments of the invention relate to systems and/or methods for heating and/or cooling a cryogenic needle, often referred to also as a cryoprobe.

### BACKGROUND

Cryoablation of tissues is a medical procedure that uses extreme low temperatures to destroy abnormal or diseased tissue. During cryoablation, the low temperature conditions are created using a sealed tip, hollow needle (cryoprobe) through which substances such as liquid Nitrogen or Argon gas are circulated. Exposure to low temperature freezes the tissue, causing the cells to die and ultimately be absorbed by the body. Cryoablation may be used to treat a variety of conditions, including cancerous tumors, abnormal heart rhythms, and certain skin conditions.

Application of cryoablation is typically performed by assistance of imaging technologies to identify a treatment site for an ablative procedure and then inserting one or more cryogenic needles (cryoprobes) into the selected site and sufficiently cooling the needles to urge the tissues surrounding the needles to reach cryoablation temperatures, typically below about −40 (minus forty) degrees Celsius.

Heating the cryogenic needles (cryoprobes) is typically performed in order to free the needles from adhesion to the frozen tissue after cryoablation, permitting rapid removal of a needle from an ablation site, thereby shortening the time required for medical procedures. Such heating of a cryogenic needle may also be useful when repositioning the needle for use at several treatment sites.

### SUMMARY

The following embodiments and aspects thereof are described and illustrated in conjunction with systems, tools and methods which are meant to be exemplary and illustrative, not limiting in scope.

In at least certain embodiments, systems and/or methods are provided for heating a cryogenic needle with a laser beam, RF wave, and/or microwave.

In addition to the exemplary aspects and embodiments described above, further aspects and embodiments will become apparent by reference to the figures and by study of the following detailed descriptions.

### BRIEF DESCRIPTION OF THE FIGURES

Exemplary embodiments are illustrated in referenced figures. It is intended that the embodiments and figures disclosed herein are to be considered illustrative, rather than restrictive. The invention, however, both as to organization and method of operation, together with objects, features, and advantages thereof, may best be understood by reference to the following detailed description when read with the accompanying figures, in which:

FIG. 1 schematically shows an embodiment in accordance with the present invention of a cryosurgical device that forms part of a system for cooling and heating a cryogenic needle of the cryosurgical device; and

FIGS. 2A to 2D schematically show embodiments in accordance with the present invention of cryogenic needles and systems for cooling and heating the cryogenic needles.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated within the figures to indicate like elements.

### DETAILED DESCRIPTION

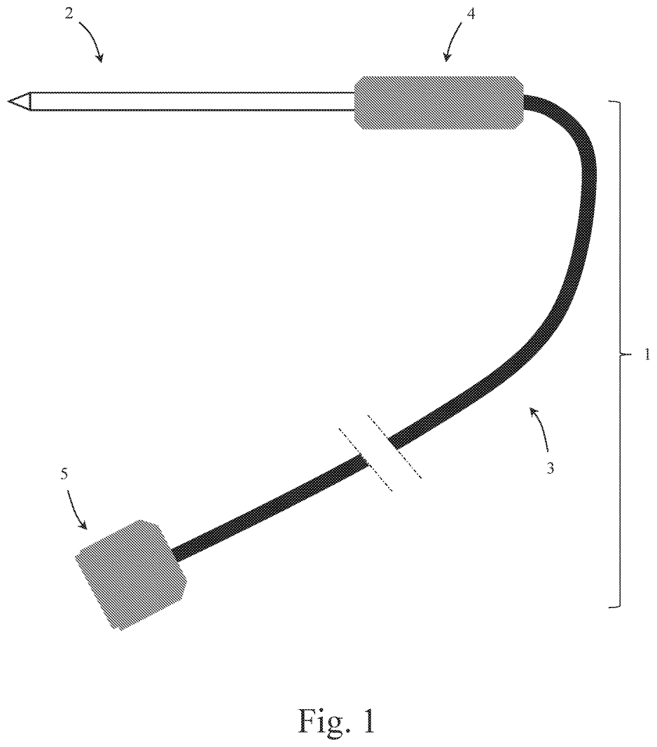

Attention is first drawn to FIG. 1 schematically showing a cryosurgical device 1 that includes a cryogenic needle 2 (often also referred to as a cryoprobe) at its distal side and a delivery line 3 that is attached to the needle in this example at a handle 4 of the device. The delivery line 3 is arranged to supply gas to the needle and evacuate the returning gas, and in various embodiments of the present invention may be configured to include additional lines (e.g. electrical wires, laser fiber, etc.) to heat the needle's tip. The cryosurgical device includes a connector 5 at its proximal side.

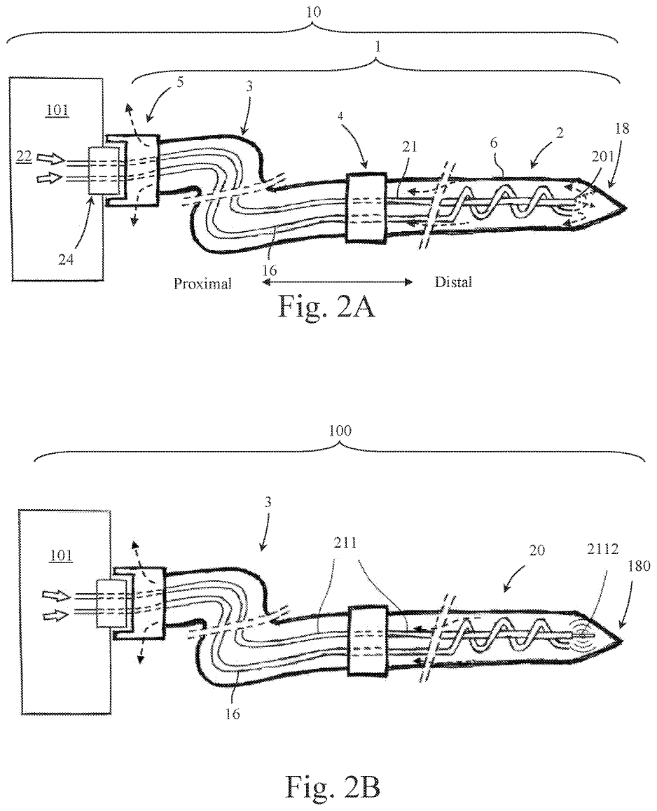

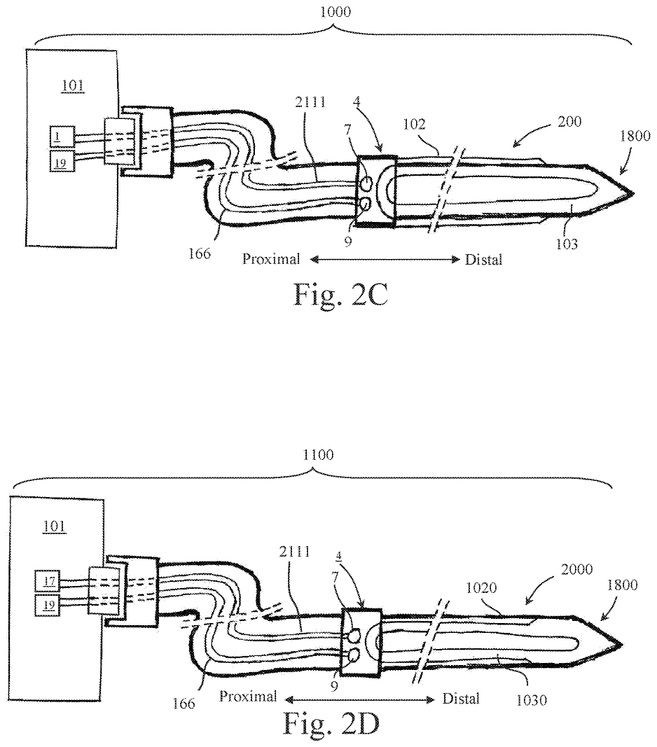

Attention is drawn to FIG. 2A showing an embodiment of a system 10 for cooling and heating a cryosurgical device 1 in accordance with an embodiment of the present invention.

The cryosurgical device in this example includes a needle 2 at its distal side and an input tube 16 that extends from its connector 5 through delivery line 3 towards needle 2. Input tube 16 as seen includes a spirally shaped section in the needle.

In certain embodiments, the system can be controlled to supply gas via input tube 16 for cooling or heating a distal tip 18 of the cryogenic needle 2. Cooling of the tip may be to cryoablation temperatures when the needle is operated in cooling mode. Heating the tip may be aimed at releasing adhesion of the needle from frozen tissues at its vicinity. Gas supplied for heating the needle may be at a relatively low pressure, of e.g. between about 300 psi and 600 psi.

In an aspect of the present invention, system 10 may include an optical fiber 21 and a laser system 22 that is configured to emit a laser beam through the optical fiber towards the needle's distal tip 18 (see ‘dotted’ arrows). The optical fiber in this example can be seen being threaded through delivery line 3 generally alongside input tube 16 and through the needle towards a distal end 201 of the fiber that is proximal to needle's distal tip 18.

In certain cases, instead of a laser beam, infrared radiation may be transported towards the needle tip 18 via an optic fiber, such as the optical fiber 21 seen in FIG. 2A.

Heat generated at the needle's distal tip 18 as a result of the laser beam (or infrared radiation) may propagate within the outer peripheral walls 6 of the needle. Furthermore, heat may be transferred from the tip 18 of the needle to the walls 6 by the flow of gas at low pressure through input tube 16.

In certain embodiments, the distal end 201 of the optical fiber may be designed and/or configured to distribute the laser energy emitted out of the optical fiber in a suitable profile/pattern rather than a precise single laser point. In one example, such suitable profile/pattern may be achieved by adding a fiber end cap at the fiber's distal end 201 in order to, inter alia, control the propagation of light. The fiber end cap may be made from a variety of materials that are substantially optically transparent, such as glass or plastic.

In certain embodiments, an optical fiber coupler may be used at an interface between the cryosurgical device's connector 5 and a needle port 24 of the system to minimize energy losses. Examples of fiber end caps and fiber couplers may be those offered by Thorlabs, Inc.

By way of a non-binding example, a specific heat capacity of an ice ball formed at the tip of cryogenic needle 12 may be about 2.108 KJ/KG-k and its weight (mainly water, small size of ø1.6 cm)=4πR\hat{ }3/3=4×π×0.8\hat{ }3/3=~2.14gr. The energy needed to heat such an ice ball from about −40 °C to about 0 °C=m×C×ΔT=2.14/1000×2.108×40=0.180 Kj=180 j. In certain embodiments where only a relatively small part of the tissue and the needle needs to be heated for releasing adhesion of the needle from frozen tissues, the energy needed may be less than 180 j. For heating an ice ball within about 10 min (600 sec), the power needed=180/600=~0.3 watt.

Attention is drawn to FIGS. 2B to 2D schematically showing further embodiments of systems 100, 1000, 1100 for heating respective cryogenic needles 20, 200, 2000. In the embodiments of FIGS. 2C and 2D, the needle shafts are embodied as a heat pipe, which are fully sealed two-phase heat transfer devices that take advantage of fluid's high latent heat of vaporization to achieve a relatively high efficient heat transfer.

In the example seen in FIG. 2C, a heating outlet 7 may be located in the needle's handle 4 and may be fed by a heating energy controller 17 which may be located in a console 101 of the system. Heat may be supplied from heating energy controller 17 towards heating outlet 7 via conventional means 2111, such as (but not only) resistor, RF antenna (and the like).

The heating outlet 7 may be arranged to transfer its temperature to a proximal end of the needle. A heat pipe formation 103 of the cryogenic needle may be configured in turn to transport the heat to the distal end of the needle to be dissipated through the needle's tip 1800. Some examples of heating outlets 7 may include (but not limited to) an electrical resistor, or a body heated by laser, by RF wave, by electromagnetic induction, or by infrared radiation.

With respect to an example of a system 100 being powered for heating by radio-frequency (RF) waves, attention is drawn to FIG. 2B. Here an RF antenna 211 is seen being channeled along the system's delivery line 3 and along the needle's shaft. The RF antenna 211 may be formed of an outer conductor and an inner conductor (wire) 2112. A dielectric material may surround the inner conductor 2112 (except an exposed distal tip thereof). The RF antenna 211 may be used for emitting radiation substantially only at its very distal exposed tip, close to the needle's tip 180, thus heating the needle's tip material. The RF wave generator may be located in the console 101. The aforementioned description with respect to the embodiment seen in FIG. 2B may also apply to a microwave powered system.

Attention is drawn back to the embodiment seen in FIG. 2C. A cooling outlet 9, possibly also located within the needle's handle 4 may be fed by a cooling energy controller 19 which may also be located within console 101. Cooling may supplied from cooling energy controller 19 towards cooling outlet 9 by conventional means 166, such as (but not only) Joules-Thompson effect, liquid nitrogen, etc. The cooling outlet 9 may be arranged to similarly transfer its temperature to the proximal end of the needle to be then transported to the distal end via the needle's heat pipe formation 103. The heat pipe formation 103 may be enrobed at its proximal part in a thermally insulating mantle 102 that is formed on an outer periphery of the generally cylindrical shaped heat pipe formation.

In the system embodiment 1100 seen in FIG. 2D, the heat pipe formation 1030 may include two sections, a first more proximal section that has a slightly smaller diameter than a second more distal section. In this example, a thermally insulating mantle 1020 may be formed about the heat pipe's first section and by that achieve a thermal insulation in the proximal part of the needle and a uniform diameter of the needle along its shaft.

In the description and claims of the present application, each of the verbs, “comprise” “include” and “have”, and conjugates thereof, are used to indicate that the object or objects of the verb are not necessarily a complete listing of members, components, elements or parts of the subject or subjects of the verb.

Further more, while the present application or technology has been illustrated and described in detail in the drawings and foregoing description, such illustration and description are to be considered illustrative or exemplary and non-restrictive; the technology is thus not limited to the disclosed embodiments. Variations to the disclosed embodiments can be understood and effected by those skilled in the art and practicing the claimed technology, from a study of the drawings, the technology, and the appended claims.

In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures can not be used to advantage.

The present technology is also understood to encompass the exact terms, features, numerical values or ranges etc., if in here such terms, features, numerical values or ranges etc. are referred to in connection with terms such as “about, ca., substantially, generally, at least” etc. In other words, “about 3” shall also comprise “3” or “substantially perpendicular” shall also comprise “perpendicular”. Any reference signs in the claims should not be considered as limiting the scope.

Although the present embodiments have been described to a certain degree of particularity, it should be understood that various alterations and modifications could be made without departing from the scope of the invention as hereinafter claimed.

1. A system for affecting a temperature of a cryogenic needle of the system, the system comprising an optical fiber and a radiation system that is configured to emit a radiation through the optical fiber towards the needle's distal tip.

2. The system of claim 1, wherein the radiation system is a laser system and the emitted radiation passing through the optical fiber is a laser beam.

3. The system of claim 1, wherein the radiation system is an infrared radiation source and the emitted radiation passing through the optical fiber is infrared radiation.

4. The system of claims 2 or 3, wherein the optical fiber being threaded through a delivery line that extends in the distal direction towards the needle.

5. The system of claim 4, wherein the delivery line is attached via a handle to the needle.

6. The system of claim 5, wherein the delivery line is arranged to supply gas to the needle and evacuate the returning gas.

7. The system of claim 4 and comprising a console and the radiation system being located within the console, wherein the delivery line extends in the distal direction away from the console towards the needle.

8. The system of claim 7 and comprising a needle port at the console and the delivery line being detachably attached to the console via the needle port.

9. The system of claim 8 and comprising an optical fiber coupler as an interface between the needle port and a connector of the delivery line that is located at the proximal end of the delivery line.

10. The system of claim 1, wherein a distal end of the optical fiber is designed and/or configured to distribute the laser energy emitted out of the optical fiber in a suitable profile/pattern rather than a precise single laser point.

11. The system of claim 10 and comprising a fiber end cap at the fiber's distal end in order to achieve the suitable profile/pattern.

12. The system of claim 11, wherein the fiber end cap being formed from materials that are substantially optically transparent, such as glass or plastic.

13. A system for affecting a temperature of a cryogenic needle of the system, the needle having a heat pipe shaft and comprising a heating outlet within a handle of the needle that is located at a proximal side of the needle.

14. The system of claim 13 and comprising a delivery line that extends in the distal direction towards the needle.

15. The system of claim 14, wherein the delivery line is attached via the handle to the needle.

16. The system of claim 14 or 15 and comprising a console and the delivery line extends in the distal direction away from the console towards the needle.

17. The system of claim 16 and comprising a heating energy controller within the console for heating the heating outlet.

18. The system of claim 17, wherein the heat pipe being arranged to transfer heat arriving at the heating outlet from the heating energy controller towards the distal end of the needle.

19. The system of claim 16 and comprising a cooling energy controller within the console for cooling a cooling outlet located within the needle's handle.

20. The system of claim 19, wherein the heat pipe being arranged to transfer the temperature from the cooling outlet towards the distal end of the needle.

21. The system of claim 13, wherein the heat pipe shaft of the needle comprising two sections, wherein a first more proximal section having a slightly smaller diameter than a second more distal section.

22. The system of claim 21 and comprising a thermally insulating mantle formed about the heat pipe's first section.

23. The system of claim 13, wherein the heat pipe shaft of the needle being enrobed at its proximal part in a thermally insulating mantle.

24. A system for affecting a temperature of a cryogenic needle of the system, the system comprising an antenna and an electromagnetic radiation source that is configured to emit electromagnetic radiation through the antenna towards the needle's distal tip.

25. The system of claim 24, wherein the electromagnetic radiation source is a radio frequency (RF) source or a microwave source.

26. The system of claim 24 or 25, wherein the antenna being threaded through a delivery line that extends in the distal direction towards the needle.

27. The system of claim 26, wherein the delivery line is attached via a handle to the needle.

28. The system of claim 26, wherein the delivery line is arranged to supply gas to the needle and evacuate the returning gas.

29. The system of claim 24 and comprising a console and the electromagnetic radiation source being located within the console, wherein the delivery line extends in the distal direction away from the console towards the needle.

30. The system of claim 29 and comprising a needle port at the console and the delivery line being detachably attached to the console via the needle port.

31. The system of claim 30, wherein the antenna comprises an outer conductor and an inner conductor wire.

32. The system of claim 31 and comprising dielectric material that surrounds the inner conductor wire.

33. The system of claim 31 or 32, wherein a distal tip of the inner conductor wire adjacent to the needle's tip being exposed.

34. A method for affecting a temperature of a cryogenic needle, the method comprising the steps of:
providing a system comprising an optical fiber and a radiation system that is configured to emit a radiation through the optical fiber towards the needle's distal tip.

35. The method of claim 34, wherein the radiation system is a laser system and the emitted radiation passing through the optical fiber is a laser beam.

36. The method of claim 34, wherein the radiation system is an infrared radiation source and the emitted radiation passing through the optical fiber is infrared radiation.

37. The method of claims 35 or 36, wherein the optical fiber being threaded through a delivery line that extends in the distal direction towards the needle.

38. The method of claim 37, wherein the delivery line is attached via a handle to the needle.

39. The method of claim 38, wherein the delivery line is arranged to supply gas to the needle and evacuate the returning gas.

40. The method of claim 37 and comprising a console and the radiation system being located within the console, wherein the delivery line extends in the distal direction away from the console towards the needle.

41. The method of claim 40 and comprising a needle port at the console and the delivery line being detachably attached to the console via the needle port.

42. The method of claim 41 and comprising an optical fiber coupler as an interface between the needle port and a connector of the delivery line that is located at the proximal end of the delivery line.

43. The method of claim 42, wherein a distal end of the optical fiber is designed and/or configured to distribute the laser energy emitted out of the optical fiber in a suitable profile/pattern rather than a precise single laser point.

44. The method of claim 43 and comprising a fiber end cap at the fiber's distal end in order to achieve the suitable profile/pattern.

45. The method of claim 44, wherein the fiber end cap being formed from materials that are substantially optically transparent, such as glass or plastic.
